(19) United States
(12) Patent Application Publication
(10) Pub. No.: US 2026/0227508 A1
(43) Pub. Date: Aug. 6, 2026

(54) METHOD AND APPARATUS FOR PACKAGING SYSTEM CIRCUITY AND A TRANSDUCER ARRAY

(71) Applicant: DarkVision Technologies Inc.

(72) Inventors: Graham Manders, North Vancouver (CA); Muhammad Imran Masud, Surrey (CA); Michael Halpenny-Mason, North Vancouver (CA); Travis Blalock, Charlottesville, VA (US); Joseph Liu, Vancouver (CA)

(21) Appl. No.: 19/147,332

(22) PCT Filed: Jan. 24, 2024
(86) PCT No.: PCT/IB2024/050652

§ 371 Date: Jul. 11, 2025

Related U.S. Application Data
(60) Provisional application No. 63/442,753, filed on Feb. 1, 2023.

(51) Int. Cl. G01S 15/89; G01S 7/52; G10K 11/00

(52) U.S. Cl. CPC G01S 15/8925; G01S 7/52046; G01S 7/52079; G01S 7/52085; G10K 11/004

(57) ABSTRACT

Various embodiments of a method and apparatus for attaching a die to a transducer array are disclosed. In some embodiments, multiple dies are provided. The dies communicatively connect to a redistribution layer, which communicates with the transducer array. The redistribution layer also communicates with a substrate holding the die, and the substrate communicates with off-chip components. The redistribution layer and substrate sandwich the die. As a result, the die can be connected as a layer backing the transducer array while allowing the die to communicate with both the transducer array and off-chip components, with electrical connections (or other communication connections) on only one side of the die. Multiple transducer packages can be combined to form arbitrarily large transducer arrays.

### RELATED APPLICATIONS

This International PCT Patent Application claims the benefit of priority to U.S. Provisional Application No. 63/442753, filed on Feb. 1, 2023. The above-referenced patent application is herein incorporated by reference in its entirety.

### TECHNICAL FIELD

The disclosed method and apparatus relate generally to systems for imaging. In particular, the disclosed method and apparatus relate to packaging circuitry used in an ultrasound imaging system and a transducer array.

### BACKGROUND

Ultrasound imaging can be used for several purposes. In some cases, such imaging is used for nondestructive testing. At times, the area to be imaged may be located in a small or difficult to access space. Consequently, it may be desirable to design an imaging head of an imaging system to be small. Such imaging heads typically have a constellation of transducers elements, each of which are capable of transmitting signals and then receiving reflections of those transmitted signals. Using one-dimensional arrays of ultrasound transducers elements in the imaging head allows the head to be small, making it easier to manipulate and fit the head into relatively small spaces. In some cases, a two-dimensional array of transducer elements produces a better image than a one-dimensional array. However, in cases in which two-dimensional imaging is desired, simply arranging transducer elements in a two-dimensional array does not necessarily result in the desired images. For example, using a scheme that activates the transducer elements to transmit and receive on a row-by-row or column-by-column basis, does not result in the same images as using a fully addressable array in which any combination of elements can be addressed to be independently activated.

Furthermore, to attain an image having the desired quality, the reflected signal received by the transducer elements should have a relatively high SNR (signal to noise ratio). Keeping the device small so that the circuitry for addressing the transducer array and processing the signals received can be close to the transducer array increases the SNR of the signal provided to the processing circuitry. However, several challenges arise when attempting to produce an architecture that has the desired array of transducers and in which the array of transducers is relatively close to the supporting circuitry.

Accordingly, it would be advantageous to provide packaging arrangement for an imaging system in which the system has a relatively large two-dimensional array of transducers that can be randomly accessed and wherein the array is located relatively close to the circuity used for addressing the transducers of the array.

### SUMMARY

Various embodiments of a method and apparatus for packaging a two-dimensional array together with the circuit used to address and control the array are described. In some of these embodiments, an SRL (Signal Redistribution Layer) interfaces several IC (Integrated Circuit) dies on one side of the SRL with a transducer array on the other side of the SRL. The SRL is an interconnection substrate having multiple sub-layers, in which each sub-layer provides conductive paths for signals being routed between the IC dies and the transducer array. In some embodiments of the disclosed apparatus, the IC dies are ASICs (Application Specific ICs). The SRL also electrically interconnects circuitry on the dies with devices that are external to the package. In some embodiments, the points of connection to external devices are located on the same side of the SRL to which the dies are coupled. In some embodiments, the SRL supports 4 dies arranged in a 2×2 array. In some embodiments, each die supports an array of 32×32 transducer elements. In some embodiments, an array of conductive bumps (commonly referred to as “solder bumps”) connect each die to the SRL. In some embodiments, the conductive bumps on the die have a smaller pitch than the pitch of the layout of transducers in the transducer array. The SRL allows the difference in the pitch of the transducers to be accommodated. In some embodiments, a flex conductor couples the SRL to an external substrate, such as a PCB (printed circuit board) or other external device or surface.

In some embodiments, the pitch of the transducers within the array is chosen based on the speed of sound in the material intended to be imaged. For example, in some such embodiments the pitch of the transducers is less than the wavelength of a wave generated by the transducer when the transducer is stimulated. In other embodiments, pitch is chosen based on the frequency, characteristics of the fluid or other material to be imaged, and amount of beam steering desired. A pitch of ½ of a wavelength may be used in some such embodiments, but up to a full wavelength can be used in other such embodiments. Furthermore, the transducer package can be ‘edgeless’ so that more than one package can be arrange into arbitrary size arrays of packages. For example, a 2×2 package can be combined in a 3×3 arrangement of packages to create a 6×6 arrangement of dies (36×256 transducer elements). A pitch matched solution does not allow this, because the inputs and outputs to application PCB or flex cables need to extend beyond the transducer.

The embodiments described herein utilize technology to drive sensor data insights. The various layers are an elevation of the application of the division of labor by comparative advantage, such that the whole stack contributes greater to signal capabilities.

### BRIEF DESCRIPTION OF THE DRAWINGS

The disclosed method and apparatus, in accordance with one or more various embodiments, is described with reference to the following figures. The drawings are provided for purposes of illustration only and merely depict examples of some embodiments of the disclosed method and apparatus. These drawings are provided to facilitate the reader's understanding of the disclosed method and apparatus. They should not be considered to limit the breadth, scope, or applicability of the claimed invention. It should be noted that for clarity and ease of illustration these drawings are not necessarily made to scale.

FIG. 1 is a simplified block diagram of an embodiment of an imaging system.

FIG. 2 illustrates details of one die.

FIG. 3 is an illustration of one embodiment of the transducer array isolated from the other components of the package of the system.

FIG. 4 shows a split portion of the cross-section A-A of the transducer array.

FIG. 5 is an exploded view of a portion of the package of the system.

FIG. 6 shows a detailed view of Section D.

FIG. 7 shows a detailed view of Section E.

FIG. 8 shows the surface of the SRL on which four dies are to be placed (dies themselves are not shown in FIG. 8).

FIG. 9 illustrates a divided cross-sectional view of another type of SRL that has a cavity with the transducer array mounted on a top side of the SRL.

FIG. 10 is a 3-dimensional illustration of the transducer array and 4 Die's mounted on the SRL.

FIG. 11 is an illustration of another embodiment a package in which a substrate has pins that allow the package to be electrically and mechanically connected to an external circuit board (not shown) or other such substrate.

FIG. 12 is an exploded perspective view of an imaging system having a “tiled” substrate on which multiple packages are placed.

FIG. 13 is an assembled perspective view of the package side of the imaging system.

The figures are not intended to be exhaustive or to limit the claimed invention to the precise form disclosed. It should be understood that the disclosed method and apparatus can be practiced with modification and alteration, and that the invention should be limited only by the claims and the equivalents thereof.

### DETAILED DESCRIPTION

A method for packaging a transducer array together with circuitry for controlling the operation of the transducer array is disclosed.

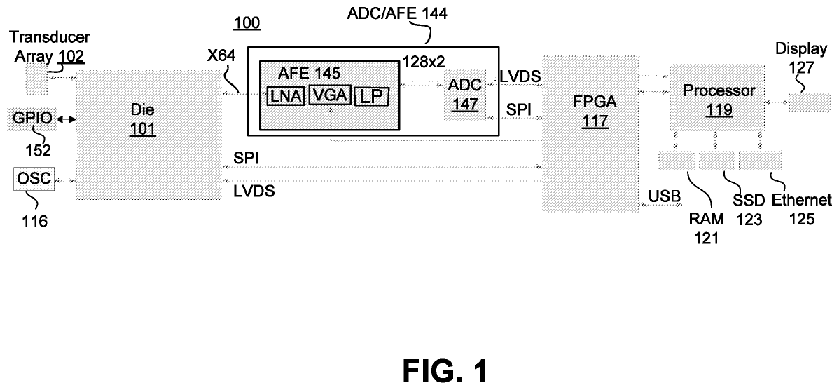

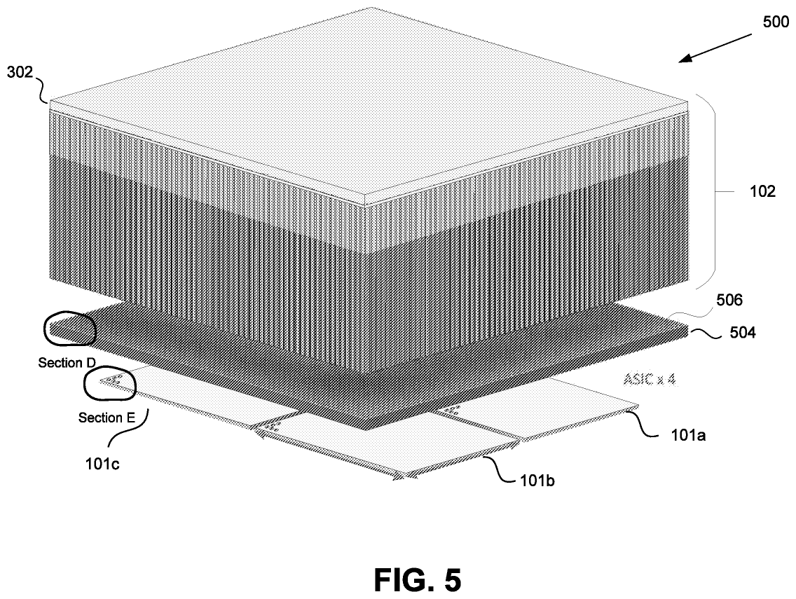

FIG. 1 is a simplified block diagram of an embodiment of an imaging system 100. It should be noted that reference designations (such as 101a and 101b, see FIG. 5) that start with the numeric value followed by an alphabetic value, and that have the same numeric value, refer to features that have essentially the same structure and function as one another. Furthermore, all such features with the same numeric value can be referenced collective using just the numeric portion of the reference designation. Accordingly, the reference 101 refers to all four of the dies 101a, 101b, 101c, 101d.

In some embodiments, the imaging system 100 includes a transducer array 102, an oscillator 116, an Analog to Digital Converter/Audio Front End (ADC/AFE) 144, a plurality of dies 101 (only one is shown for the sake of simplicity), a General Purpose Input/Output (GPIO) port 152 to the die 101, a Field Programmable Array (FPGA) 117, a processor 119, random access memory (RAM) 121, a Solid State Drive (SSD) 123, an Ethernet port 125 and a display 127. The ADC/AFE 144 comprises an ADC 147 and an AFE 145. The AFE 145 includes a Low Noise Amplifier (LNA), a Variable Gain Amplifier (VGA), and a Low Pass filter (LP). Dies 101 may be used by OEM (Original Equipment Manufacturer) integrators. In some embodiments, the die 101 allows imaging to be performed through two materials (such as a wedge interface plate or water) and allows for imaging in materials ranging from fluids to steel.

Some embodiments of the presently disclosed system 100 use the transducer array 102 to generate and transmit an acoustic wave into a target region, receive a reflection of the transmitted signal and create an image based on the received reflection. Upon receiving the reflected signals, the transducer array 102 converts the received signals to a format that can be analyzed (e.g., converts received acoustic signals to electrical signals). In some embodiments in which ultrasound pulses are generated and transmitted, the transducer array 102 comprises a two-dimensional X×Y element array of piezoelectric transducer elements (where X or Y can be 32, 64 or any other larger number which is a multiple of 32). In some embodiments, the elements are made of lead zirconate titanate Pb[ZrxTil-x]O3 (0≤x≤1).

A delay profile is generated that determines the shape of the waveform that is transmitted by the composite of the transmitting elements of the transducer array 102. As used in this disclosure, a “waveform” is the acoustic wave generated by exciting one or more of the transducer elements. In some cases, a single pulse is applied to each transducer to form the waveform. In other cases, a series of pulses is applied to one or more of the transducer elements in rapid succession. Exciting the transducer elements with a single pulse creates a sharper image. However, using a series of pulses transmitted by each transducer element reduces the signal-to-noise ratio in the final image.

Accordingly, in some embodiments, all of the transducer elements or some subset of transducer elements may be excited to transmit acoustic pulses and thus form an acoustic waveform that will propagate into the image region. In some embodiments, a user indicates to the processor 119 parameters related to how the image is to be taken, such as the shape of the waveform to be transmitted into an imaging region and the number of angles involved in each set of transmissions. Alternatively, the user may specify a less detailed plan for imaging a particular object or image region, in which case, specifics about the shape of the waveform and other specifics, such as the number of angles, etc., are determined automatically by the processor 119.

In some embodiments, once the processor 119 selects a particular waveform and/or set of waveforms to be transmitted into the image region, the processor 119 conveys parameters to the FPGA 117 that can be used to generate a delay profile that controls when each transducer element of the transducer array 102 will be excited. The FPGA 117 uses that information to “stage” the set of waveforms to be transmitted. That is, the FPGA 117 uses the information that the processor 119 provides to determine how many times the transducer array 102 will transmit a waveform and which elements of the transducer array 102 are to be excited for each such transmission. As part of that process, the FPGA 117 also coordinates the timing between the various dies, each of which is responsible for controlling the excitation of a subset of the entire transducer array 102.

The FPGA 117 provides signals to each die 101 based on the information provided by the processor 119 to the FPGA 117 to allow each die 101 to determine when to transmit waveforms and to establish a delay profile to be applied to those elements of the transducer array 102 associated with each particular die 101 when transmitting each such waveform.

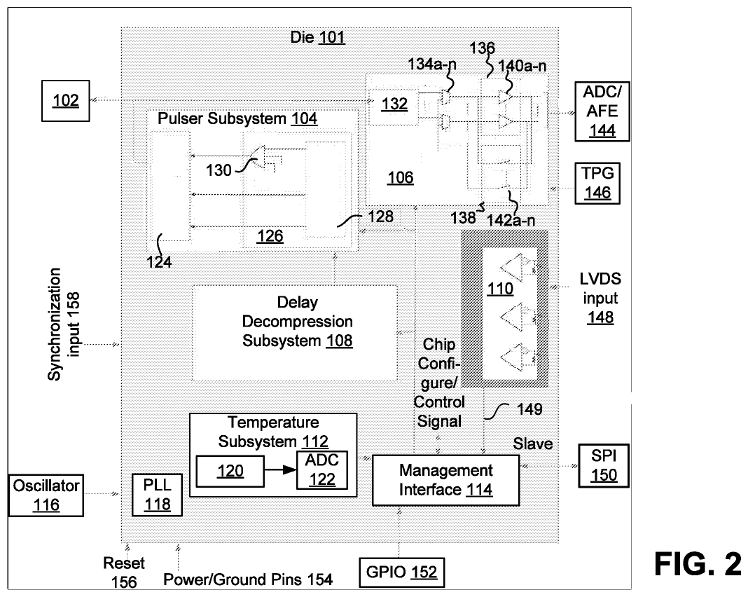

FIG. 2 illustrates details of one die 101. Each die 101 comprises a pulser subsystem 104, a receive switch subsystem 106, a delay decompression subsystem 108, a Low-Voltage Differential Signaling (LVDS) subsystem 110, a temperature subsystem 112, a management interface 114 and an optional Phase Lock Loop (PLL) 118. The principal function of the set of dies 101 is to cause the transducer array 102 to generate pulses and to capture the responsive reflections of those pulses. The resulting captured data is then provided to the processor 119 through the FPGA 117. It will be understood that in some embodiments, there are several dies 101 and that each die 101 is controlled by the FPGA 117 based on information provided to the FPGA 117 by the processor 119. Each such die 101 operates essentially the same. Therefore, only one such die is explained in detail for clarity and brevity.

The die 101 receives power and is connected to ground by power/ground pins 154. The die 101 can be reset by reset pins 156. The synchronization signal 158 synchronizes the die 101 with other dies. The temperature subsystem 112 determines the temperature of the die 101. The temperature subsystem 112 generates a signal that can then be used for temperature compensation to reduce the effects of the temperature distortion. Each die 101 is associated with and controls the operation of one section of the transducer array 102. In some embodiments, 4 dies 101 are provided, each being responsible for controlling a subset of the transducer elements of the transducer array 102. In some embodiments, the transducer array 102, the LVDS subsystem 110, and the oscillator 116 are always on. In contrast, the pulser subsystem 104, the receive switch subsystem 106, the delay decompression subsystem 108, and the temperature subsystem 112 may be turned off to save power when those components are not needed.

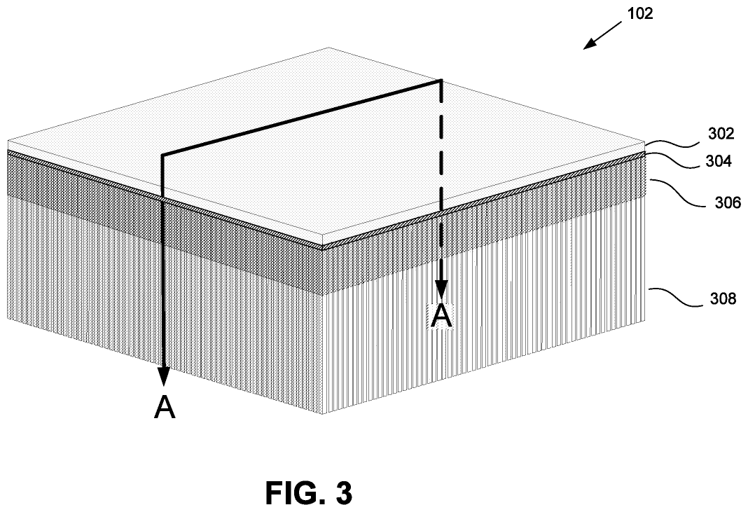

FIG. 3 is an illustration of one embodiment of the transducer array 102 isolated from the other components of the package of the system 100. It should be noted that the transducer array 102 shown is merely one example of a transducer array architecture. There are several other architectures that may be used to produce the transducer array 102. The transducer array 102 comprises a matching layer 302, a common electrode 304, a transducer material layer 306, and a backing layer 308. In some embodiments, the transducer material layer comprises PZT (lead zirconate titanate) material. However, PZT material is merely one example of the material that can be used to form the transducer.

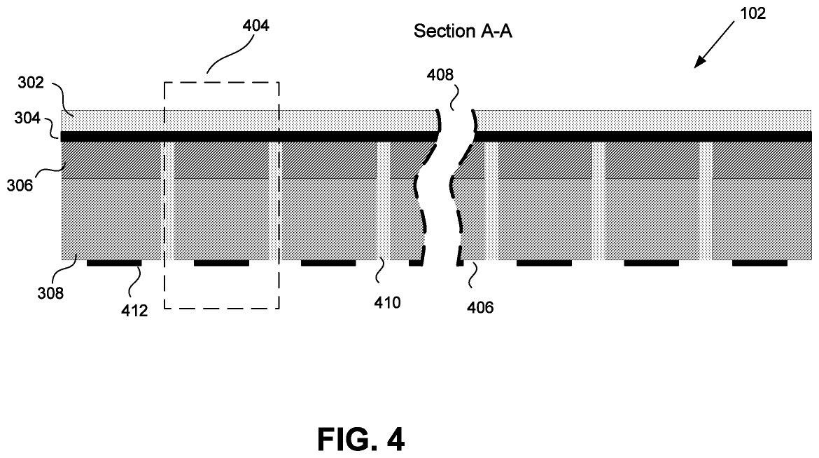

FIG. 4 shows a split portion of the cross-section A-A of the transducer array 102. Six complete transducers 404 of the transducer array 102, and two partial transducers 406 are shown. In some embodiments, the transducer array 102 is a matrix of transducers. Cross section A-A is broken at the break 408 to allow the figure to conform to the size of the page, while still showing the relevant details. In some embodiments, transducers 404 are each separated from adjacent transducers 404 by an insulating material 410 that fills dicing cuts between portions of the transducer material layer 306 and the backing layer 308 that form each transducer 404. “Dicing” is the process of cutting a block of material into individual elements. The cuts that are made to separate the material into the individual elements are referred to as “dicing cuts”.

The transducer material layer 306 lies between the common electrode 304 and the backing layer 308. The matching layer 302 resides on top of the common electrode 304. A transducer electrode 412 is formed on the bottom of the backing layer 308. The transducer electrodes 412 provide an interface to allow signals to be applied to each of the transducers 404. Each transducer 404 can be excited to generate an acoustic wave by applying a voltage between the transducer electrode 412 for the particular transducer 404 and the common electrode 304. The electrical signal applied to the transducer electrode 412 is transmitted through the backing layer 308. Accordingly, conductive paths are formed through the backing layer 308.

The backing layer 308 acoustically dampens the response of the transducer. This shortens the pulse. The result is a higher bandwidth pulse. Higher bandwidth pulses provide better resolution. The matching layer 302 reduces the difference (i.e., mismatch) between the acoustic impedance of a target region and the acoustic impedance of the transducer material layer 306 within the transducers 404. In cases in which the system is to be used to image a target region through water, the matching layer 302 would be used to match the acoustic impedance of the water medium to that of the transducer material layer 306.

It should be noted that several sub-layers (not shown) within the matching layer 302 may be used to provide a transition between the material to be imaged and the transducer material layer 306. While the matching layer 302 is shown to be uniform in depth, in some embodiments, the matching layer 302 is formed to conform to the shape of the region or material to be imaged, or through which the image will be attained. For example, in some such embodiments, the matching layer 302 is formed as a wedge (not shown).

When the reflected wave is received at the transducer material layer 306, the PZT material generates an electrical signal. The electrical signal is conducted from the PZT material of the transducer material layer 306, through the backing layer 308 and to the transducer electrode 412.

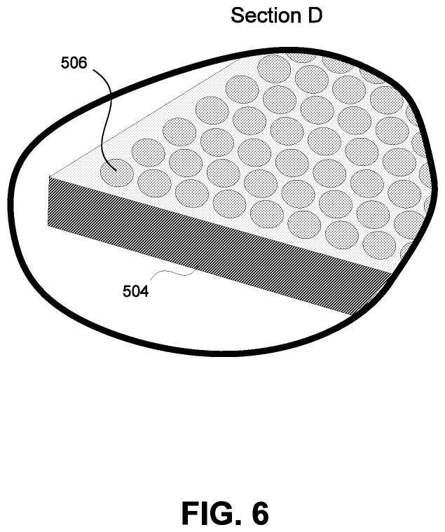

FIG. 5 is an exploded view of a portion of the package 500 of the system 100. The transducer array 102 is mounted on an SRL (Signal Redistribution Layer) 504. The SRL 504 has a first side having an array of contact points 506, each of which is aligned with a corresponding transducer electrode 412 (see FIG. 4) of the transducer 102. Accordingly, upon mounting the transducer 102 on the SRL 504, electrical contact is made between each of the transducer electrodes 412 and a corresponding one of the contact points 506. The left-most corner of the SRL 504 is highlighted as Section D. FIG. 6 shows a detailed view of Section D.

In the embodiment shown, four dies 101 are mounted on the side of the SRL 504 opposite the side on which the transducer array 102 is mounted. In this case, three dies 101a, 101b, 101c are visible in the FIG. 5. A fourth die is hidden behind the SRL 504 and the transducer array 102. Each such die 101 is essentially the same as each other die 101.

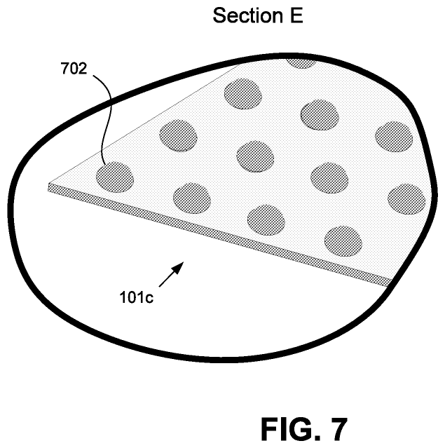

The left-most corner of one of the dies 101c is highlighted as Section E. FIG. 7 shows a detailed view of Section E. A grid of solder micro-bumps 702 reside on the surface of the dies 101. In some embodiments, solder micro-bumps 702 are provided on each of the dies 101 spaced at a distance between centers of adjacent solder micro-bumps 702 along the width of the die 512a and spaced apart along the length of the die by a distance that may be either longer or shorter than the distance between adjacent bumps along the width. The solder micro-bumps 702 are each electrically coupled to circuitry fabricated within the die 101. The solder micro-bumps 702 allow such circuitry in the die 101 to be connected to devices outside the die 101 through the SRL 504. It should be noted that in FIG. 5, only some such micro-bumps 702 are shown for the sake of simplicity. However, in some embodiments, such micro-bumps 702 populate the ensure surface. In other embodiments, fewer micro-bumps 702 may be present.

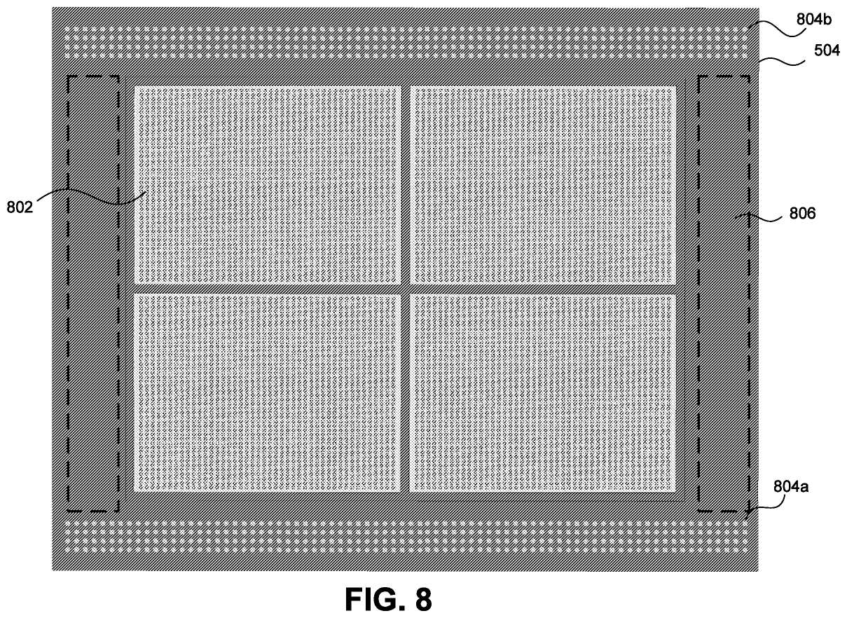

FIG. 8 shows the surface of the SRL 504 on which four dies 101 (dies not shown in FIG. 8) are to be placed. In the embodiment shown, the area where a die 101 is to be placed has a plurality of lower contacts arranged in a lower contact array 802 in which the location of each of the lower contacts corresponds to the location of one of the solder micro-bumps 702 on a die 101 that is to be placed over the lower contact array 802. Flowing the solder of the solder micro-bumps 702 provides a secure mechanical and electrical connection between the die 101 and the SRL 504. It should be noted that in other embodiments, other means can be used to secure the die 101 to the SRL 504 and provide the electrical connections between the two. Furthermore, in some such embodiments, the means for mechanically securing the die 101 to the SRL 504 may be distinct from the means for providing electrical connections between the two. Also provided on the same side of SRL 504 are two additional contact arrays 804, each located between the edge the SRL 504 and at least one of the lower contact arrays 802. In some embodiments, the additional contact arrays 804 are each essentially rectangular with the length of each of the additional contact arrays 804 running parallel to the edge of the SRL. The SRL 504 also provides for mounting passive or active devices, such as capacitors or other components (not shown) that are electrically coupled through the SRL 504 to circuitry on either the dies 101 or the transducer array 102. In some embodiments, such devices 806 reside in an area like in 806.

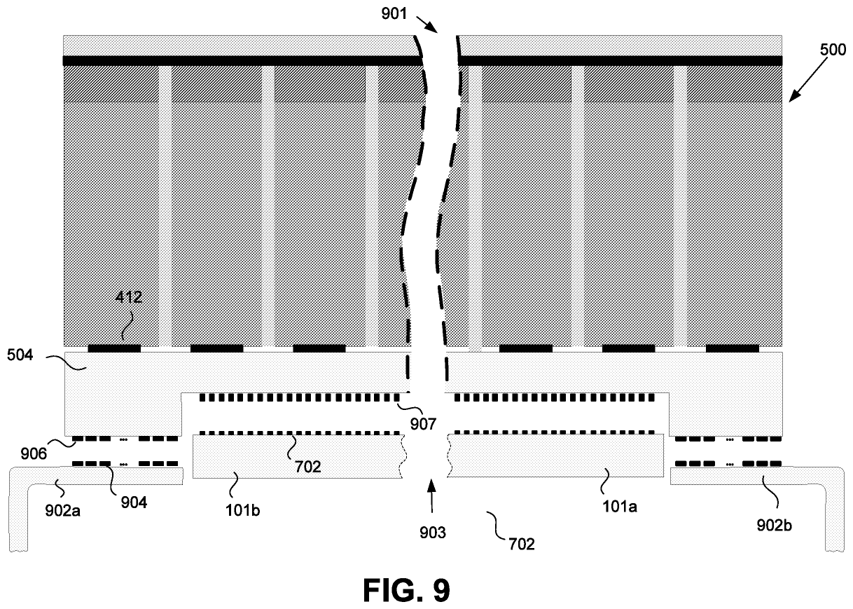

FIG. 9 illustrates a divided cross-sectional view of the SRL 504 with the transducer array 102 mounted on a first side of the SRL 504. A right half and a left half of the package 500 are shown divided (separated by a gap 901) to enable a more detailed view of the package 500. Flex cables 902 for coupling to the package 500 are also shown. It should be understood that the gap 901 is merely an illustrative device and does not exist in the package 500. Likewise, a gap 903 is shown between a portion of a first one of the dies 101b and a portion of a second one of the dies 101a. Flex cables 902 having contact arrays of mating contact points 904 that are complementary to the contacts 906 of the contact arrays 804 on the SRL 504 are shown (unmated).

Mating the contacts 904 of the contact array of the one of the flex cables 902 to the contacts 906 of the contact array 804 establishes both electrical and mechanical connections between the SRL 504 and the flex cable 902. The contacts 906 of the contact arrays 804 are electrically coupled to various other contacts of the SRL 504. These paths are established to provide an electrical path from the conductors (not expressly shown) of the flex cables 902 to various other contacts of the SRL 504. In some cases, several conduction layers (not shown) provided within the SRL 504 form the paths that route signals appropriately from the upper contacts 506 of the SRL 504 to contacts 907 of the lower contact array 802 and from contacts 907 of the lower contact array 802 to contacts 906 of the contact array 804. Accordingly, electrical connections through the SRL 504 allow the transducer array 102, the dies 101, and the flex cables 902 to be appropriately coupled to one another. In addition, in some embodiments, a recess 908 is formed in the SRL 504 to allow the dies 101 to reside securely within the recess 908. As shown, neither the dies 101 nor the flex cable 902 are mated to the SRL 504 in order to show the contacts 904, 906, 907 and the solder micro-bumps 702 more clearly.

When the dies 101 are mounted on the SRL 504 (two of which 101a, 101b are visible in the figure), the SRL 504 provides an electrical and mechanical interface between the transducer array 102 and the dies 101. Note that while there are four dies 101 connected to the SRL 504, there are only two flex cables 902 in the embodiment shown. Other embodiments may be fewer or more such flex cables.

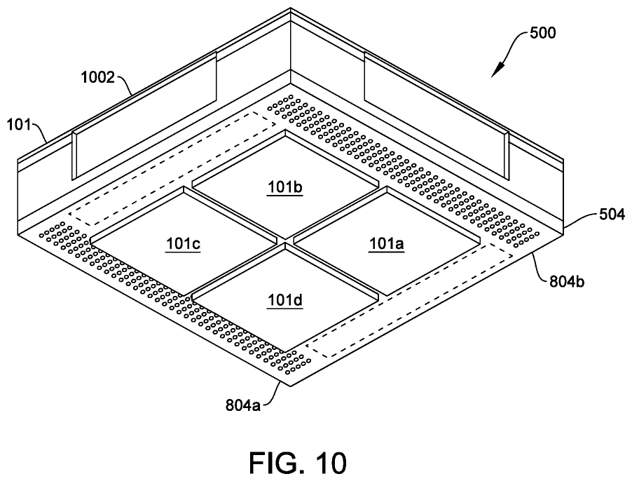

FIG. 10 is a 3-dimensional perspective view of the transducer array 102 mounted on the SRL 504. In some embodiments, side panels 1002 provide structural support. It can be seen that the SRL 504 provides a means by which to interface the contacts on the dies 101, which have smaller pitch (i.e., spacing between contacts) to the transducer electrodes 412 (see FIG. 4), which have a pitch that is greater than the contacts of the dies 101. As such, the SRL 504 acts as a redistribution layer, in that the SRL 504 redistributes the signal from the backing layer 308 to the signals from the dies 101 (from an array of one pitch to an array of another pitch). In some embodiments, the SRL 504 includes 20 to 50 conduction layers for routing the signals. In other embodiments, more or less conduction layers may be provided. The dies 101 receive the incoming signal and produce output signals. The output signals of the dies 101 are sent back into the SRL 504 (along different electrical paths than the incoming signals). The flex cables attached to the SRL 504 connect the dies 101 with the FPGA 117 (see FIG. 1). In some embodiments, the FPGA 117 is connected directly to the SRL 504, and the output signals are sent directly from the SRL 504 to FPGA 117. In some embodiments, the SRL 504 is made from Low-Temperature Cofired Ceramics (LTCCs).

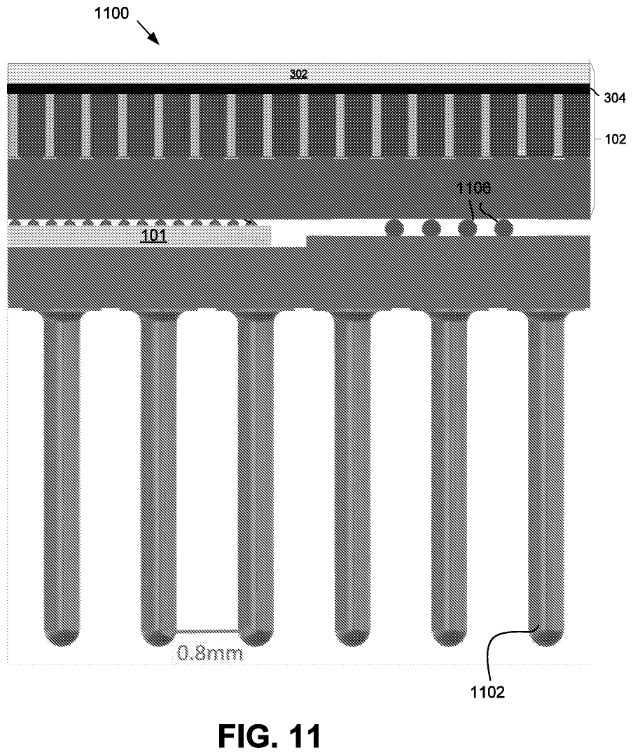

FIG. 11 is an illustration of another embodiment a package 1100 in which a substrate 1101 has pins 1102 that allow the package 1100 to be electrically and mechanically connected to an external circuit board (not shown) or other such substrate, as opposed to the flex cables previously discussed. Furthermore, rather than having the dies 101 recessed into an SRL, as is the case in the SRL 504 previously described, an SRL 1104 has a planar surface on which the dies 101 are mounted. Contacts 1106 are provided to mechanically and electrically interface the SRL 1104 to the pin bearing substrate 1102.

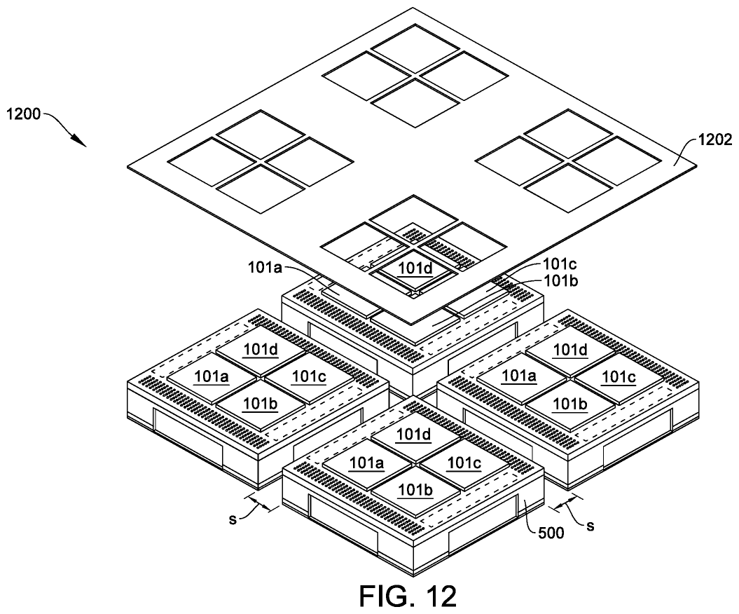

FIG. 12 is an exploded illustration of an imaging system 1200 having a “tiled” substrate 1202, such as a PCB (printed circuit board), on which multiple packages 500 are placed. This substrate 1202 is the back of the imaging system, away from the target being imaged. The substrate contacts the additional contact arrays 804 of each package to route signals from all ASICs 102 to some further circuit, processor or connector. Windows through the substrate are aligned over the ASICs to allow cooling of the ASICs. Accordingly, the imaging system 100 can be scaled to allow larger arrays of transducers working together. While the embodiment shown has four packages 500, each having four dies 101, the number of packages 500 and the number of dies 101 per package 500 may be either greater than four, or less than four. Furthermore, the figures are not necessarily drawn to scale. In some embodiments, in at least two of the packages 500, the SRL 504 has a footprint that is essentially the same as the footprint of the transducer array 102 within the same package as the SRL 504. That is, the physical exterior dimensions of the SRL 504 and the transducer array 102 within the same package are essentially the same.

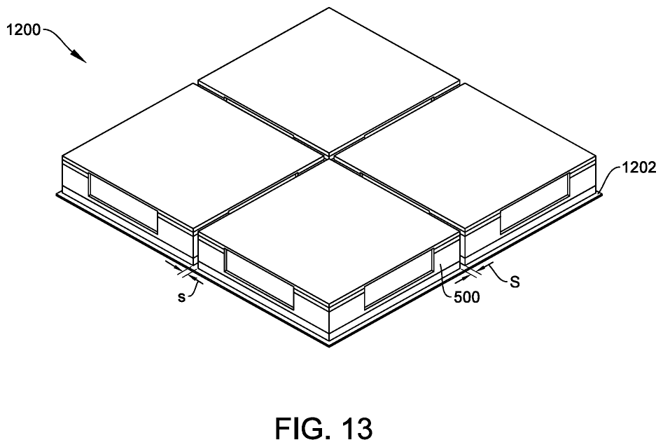

FIG. 13 is an illustration of the package side of the imaging system 1200 (i.e., the opposite side from that predominantly showing in FIG. 12). Packages 500 are shown spaced apart by a distance “s”, however, the packages 500 may be placed close to one another. In light of the fact that the footprint of the transducer array 102 is similar to the footprint of the SRL 504, the distance ‘s’ can be very small. The ASIC is designed to have a pitch much smaller than the transducer array (e.g. 150 um vs 350 um) and then use the SRL to achieve this similar footprint. The reduced pitch makes room for the additional array of I/O contacts 804 that can be routed to an application PCB or flex circuit using a 40-50 layer substrate.

These tiled imaging systems are tiled at the package level rather than the chip level. The similar footprint with edgeless design allows the imaging system to scale in both dimensions, that is, to create a true 2D array of tiled packages 500 because packages can abut each other on all sides, rather than being limited to only being tiled on 1, 2, or 3 sides of a package. Said another way, the tiled imaging system can extend by more than two packages in each dimension (e.g. 3×3, 4×4, 4×3 . . . 10×10).

The PZT material has the individual dipole moments aligned. Aligning the PZT material is achieved by applying a high voltage across the electrodes of the PZT for a time duration from ~180s to up to ~3 minutes. In some embodiments, PZT polling is performed once, initially, as part of an assembly process. In some embodiments, PZT polling is performed once after an ASIC reflow is performed. The PZT polling may also be done at other times when die 101 has experienced higher temperatures.

When polling, high voltage is applied to the common electrodes 304 and the die 101 (reversing roles of electrodes for polling only). Also, inputs/outputs are grounded when the on/off signal is asserted. For normal operation, the common electrode 304 is grounded.

Although the disclosed method and apparatus is described above in terms of various examples of embodiments and implementations, it should be understood that the particular features, aspects, and functionality described in one or more of the individual embodiments are not limited in their applicability to the particular embodiment with which they are described. Thus, the breadth and scope of the claimed invention should not be limited by any of the examples provided in describing the above disclosed embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide examples of instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the disclosed method and apparatus may be described or claimed in the singular, the plural is contemplated to be within the scope thereof unless limitation to the singular is explicitly stated.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described with the aid of block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A system comprising:
a transducer array having a plurality of transducer elements, each transducer element having a transducer electrode;
an SRL (substrate redistribution layer) having a plurality of upper contacts, each of the upper contacts configured to be mated to a corresponding one of the plurality of transducer electrodes and at least one lower contact array comprising a plurality of lower contacts;
at least one die having a plurality of electrical connections, each configured to be mated to a corresponding one of the lower contacts of the SRL.

2. The system of claim 1, wherein the SRL further comprises at least one additional contact array configured to be connected to a flex cable.

3. The system of claim 2, wherein the at least one additional contact array resides on the same side of the SRL as the lower contact array, the additional contact array residing between an edge of the SRL and at least one of the lower contact arrays.

4. The system of claim 1, further comprising a plurality of passive elements mounted to the SRL on the same side of the SRL as the lower contact array, the passive elements residing between a first edge of the SRL and at least one of the lower contact arrays.

5. The system of claim 4, wherein the SRL further comprises at least one additional contact array configured to be connected to a flex cable residing on the same side of the SRL as the lower contact array, the additional contact array residing between an edge of the SRL adjacent the first edge and at least one of the lower contact arrays.

6. The system of claim 1, further comprising a backing layer and a transducer material layer, the backing layer situated between the transducer material and the SRL.

7. The system of claim 1, further comprising a matching layer configured to reduce differences in an acoustic impedance between the transducer material layer and the acoustic impedance of the material in the target region.

8. The system of claim 1, wherein the lower contacts of the lower contact array are configured such that all of the lower contacts lie within a footprint of the transducer array.

9. A two-dimensional ultrasound imaging hardware tiled system in which a plurality of systems of claim 87 are tiled together.

10. The two-dimensional ultrasound imaging hardware of claim 9, wherein the SRL has a footprint and the transducer array has a footprint, the footprint of the SRL and the footprint of the transducer array being essentially the same.
